(19) United States
(12) Patent Application Publication
(10) Pub. No.: US 2022/0244839 A1
(43) Pub. Date: Aug. 4, 2022

(54) USER INTERFACE FOR A THERAPY DEVICE

(71) Applicant: ResMed Pty Ltd, Bella Vista, NSW (AU)

(72) Inventors: Benjamin John LEAVENS, Woollahara (AU); Vladimir ILIC, Sydney (AU)

(73) Assignee: ResMed Pty Ltd, Bella Vista, NSW (AU)

(21) Appl. No.: 17/610,102

(22) PCT Filed: May 12, 2020
(86) PCT No.: PCT/AU2020/050467

§ 371 Date: Nov. 9, 2021

(30) Foreign Application Priority Data
May 13, 2019 (AU) 2019901626

(51) Int. Cl. G06F 3/04847; A61M 16/00; G06F 3/0482; G06F 3/0488; G06F 3/0485; G06F 3/0362; G06F 3/02; G16H 40/63

(52) U.S. Cl. CPC G06F 3/04847; A61M 16/022; G06F 3/0482; G06F 3/0488; A61M 2205/505; G06F 3/0362; G06F 3/02; G16H 40/63; G06F 3/0485

(57) ABSTRACT

A medical therapy device may include a user interface (6100) such as a combined touchscreen/dial user interface, for input of operation parameters. The user interface may include a dial and a touchscreen to display a subset of a list of items. Each item may represent a parameter of the medical device. The displayed subset may include a pre-selected item that may provide a pre-selection indication for a particular item. Rotation of the dial may move the pre-selection indication so it may change between items on the displayed subset. In response to a swipe gesture on the touchscreen, the displayed subset may scroll through the list. Also, the swipe gesture may cause pushing of the pre-selection indication against a boundary of the display such as in the direction of the swipe gesture such that the item associated with the pre-selection indication remains part of the displayed subset.

### 1 CROSS-REFERENCE TO RELATED APPLICATIONS

This application claims the benefit of Australian Provisional Application No. 2019901626, filed 13 May 2019, the entire disclosure of which is hereby incorporated herein by reference.

### 2 BACKGROUND OF THE TECHNOLOGY

### 2.1 Field of the Technology

The present technology relates to user interfaces for medical therapy devices and in particular to user interfaces that may combine the features of dial and touchscreen interfaces.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, ninth edition, published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and high flow therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open interface at controlled flow rates similar to, or greater than peak inspiratory flow rate. HFT has been used to treat OSA, CSR, COPD and other respiratory disorders. One mechanism of action is that the high flow rate of air at the airway entrance improves ventilation efficiency by flushing, or washing out, expired CO₂ from the patient's anatomical deadspace. HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT). Doctors may prescribe a continuous flow of oxygen-enriched air at a specified purity (from 21%, the oxygen fraction in ambient air, to 100%) at a specified flow rate (e.g., 1 litre per minute (LPM), 2 LPM, 3 LPM, etc.) to be delivered to the patient's airway.

Oxygen concentrators have been in use for about 50 years to supply oxygen for respiratory therapy. Such devices may be large stationary oxygen concentrators or portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen, or high flow oxygen therapy (HFOT).

### 2.2.3 Respiratory Therapy Systems

These respiratory therapies may be provided by a respiratory therapy system or device. A respiratory therapy system may comprise a Respiratory Therapy Device (RT device), a patient interface, an air circuit, a humidifier, and an oxygen source.

### 2.2.3.1 Respiratory Therapy (RT) Device

A respiratory therapy (RT) device is configured to generate a flow of air for delivery to an interface to the airways. The flow of air may be pressure-controlled (for respiratory pressure therapies) or flow-controlled (for flow therapies such as HFT). Thus, RT devices may act as respiratory pressure therapy (RPT) devices and/or respiratory flow therapy devices. Examples of RPT devices include CPAP devices and ventilators.

### 2.2.3.2 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. For pressure therapies, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For flow therapies such as nasal HFT, the patient interface may be configured to insufflate the nares but specifically to avoid a complete seal. One example of such an unsealed patient interface is a nasal cannula.

### 2.2.3.3 Air Circuit

An air circuit is a conduit or a tube constructed and arranged to allow, in use, a flow of air to travel between two components of a therapy system such as the RPT device and the patient interface. In some cases, there may be separate limbs of the air circuit for inhalation and exhalation. In other cases, a single limb air circuit is used.

### 2.2.3.4 Humidifier

Delivery of a flow of air without humidification may cause drying of the airways. The use of a humidifier with an RPT device produces humidified air that minimizes drying of the nasal mucosa and increases patient airway comfort. In addition, in cooler climates, warm air applied generally to the face area in and about the patient interface is more comfortable than cold air. Humidifiers therefore often have the capacity to heat the flow of air as well as humidifying it.

### 2.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufactured large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption.

### 2.2.3.6 Medical Therapy Device User Interface

As will be evident from such respiratory related examples discussed herein, medical therapy devices are devices designed and configured to deliver, or form part of a system for delivering, a medical therapy to treat a patient's condition. An example of a respiratory related medical therapy device is a ventilator, which is designed and configured to deliver ventilation therapy to a patient with a respiratory condition.

Medical therapy devices are of ever-increasing complexity. One reason is that medical therapies are rarely one-size-fits all, but instead require a greater or lesser degree of customisation of the control parameters utilized by a controller of the device to adapt a basic therapy, such as ventilation, to the needs of a particular patient. Thus, such parameters of the controller may typically involve settings regarding the controller's operation of some motorized component, heating element and/or other servo-controlled device such as a pressure generator, etc. Typically, customisation takes the form of choosing values for such parameters for the therapy that the device is designed to deliver. In the example of ventilation therapy, the parameters may include any one, more or all of maximum airway pressure, minimum airway pressure, maximum inspiratory time, and backup rate, among others, such as those mentioned herein. The therapy device therefore needs to be configured with an interface through which a user can review the parameters whose values may be, or need to be, chosen, select one or more of the reviewed parameters in turn, and enter a chosen value for each selected parameter. This is a technical problem that is not always an easily solvable one for various reasons, including for example the complexity of the nature of the parameter, which are not always easily understood, the display and input limitations of medical devices, and/or the nature of a user or patient, who may be infirm.

Typical user interfaces comprise a graphic display such as an LCD screen configured to display parameters, and possibly default, current, or potential values for those parameters, and some means for selecting one of the displayed parameters and editing values for the selected parameter. Such a selecting device could be a dial, a button, or a touch-sensitive area such as a trackpad. Touchscreen displays are displays that are also touch-sensitive.

The number of parameters for a complex therapy such as ventilation is potentially large, of the order of dozens. However, the number of parameters that may be legibly displayed on a medical therapy device display is often much smaller than the number of parameters whose values may be chosen. In this situation, only some subset of the parameters may be displayed at any time. Interfaces therefore need to provide some way of changing the currently displayed subset to allow the user access to all parameters, as well as enabling selection of a displayed parameter and editing of a value for a selected parameter.

Dials typically allow rapid navigation between neighbouring items displayed in an ordered sequence such as a list. One item on the list is the “pre-selected” item, also referred to as the item “in focus”. A turn of the dial through a certain predefined angle shifts the pre-selected item to a neighbouring item in the displayed sequence in a direction dependent on the direction in which the dial was turned (e.g. upwards for a clockwise turn). If the dial is turned to pre-select an item outside the currently displayed subset, the currently displayed subset changes to include the newly pre-selected item, in an operation known as “scrolling”. In a “scroll”, one or more items move off one end of the display (and out of the displayed subset), while an equal number of items move into the display from the other end (and into the displayed subset). The remaining items in the displayed subset shift away from the newly displayed item(s) to make room for the newly displayed item(s).

By pressing a button, the user may select the currently pre-selected item. If the items on the list represent parameters of a therapy, selection of an item serves to select the corresponding parameter to enable editing of a value for the parameter. In some user interfaces, the button and the dial may be combined into a single control that may be both rotated and pressed, referred to as a “button dial”. Editing a parameter value for the associated parameter of a selected item may be accomplished by further rotation of the dial to navigate the “focus” through a sequence of values, and pressing of a button to choose the value currently in focus.

A touchscreen, by contrast, does not require pre-selection or “focus”, since items in the displayed list are randomly accessible by simply touching any displayed item. To accomplish scrolling, some touchscreens support an action known as “swiping”. In response to an extended touch that moves in a vertical direction, a gesture known as a “swipe”, the displayed subset scrolls with a pace and direction that keeps pace with the moving touch. Some touchscreen scrolls are “inertial”, meaning the scroll continues for some time after the swipe is complete, mimicking the motion of a moving object that decelerates gradually when a force is withdrawn, rather than stopping immediately. Inertial scrolls are particularly suitable for touchscreen displays of limited area, since a user can scroll through many items with one gesture of sufficient vigour rather than having to repeatedly swipe in the same direction.

It is beneficial for devices intended for hospital use to have dial interfaces. This is because in a hospital environment, users may not be able to activate touchscreens reliably, for example if their hands are wet or encased in rubber gloves. However, users of medical therapy devices often find touchscreen interfaces more convenient or familiar, particularly in light of the ubiquity of touchscreen-driven personal devices such as smartphones.

Therefore, it may be desirable to develop new data input interfaces to improve parameter selection for operation of medical devices.

### 3 BRIEF SUMMARY OF THE TECHNOLOGY

The present technology is directed to a user interface, such as a touchscreen/dial user interface, for a medical therapy device and methods of controlling such interfaces in response to user gestures.

According to one aspect of the present technology, when a swipe operation is performed on a displayed subset of a list of items including a pre-selected item that would cause the pre-selected item to move beyond a boundary of the display, the pre-selection moves to a boundary item of the displayed subset, which on the display looks as if the pre-selection is being pushed against the boundary of the display. The result is that a pre-selected item remains visible on the display in the location the user expects.

Some versions of the present technology may include a user interface for a medical therapy device. The user interface may include a touchscreen configured to display a subset of a list of items, each item may represent a parameter to operate the medical therapy device. One item in the displayed subset may be associated with a pre-selection indication. The user interface may include a dial configured to be rotated. The pre-selection indication may move from an item to an adjacent item on the displayed subset in response to a rotation of the dial by a predetermined angular increment. In response to a swipe gesture on the touchscreen, the displayed subset may scroll through the list and the pre-selection indication may push against a boundary of the touchscreen in the direction of the swipe gesture such that the item associated with the pre-selection indication remains part of the displayed subset.

In some implementations, the pre-selection indication may push against an upper boundary of the touchscreen in response to an upward swipe gesture. The pre-selection indication may push against the lower boundary of the touchscreen in response to a downward swipe gesture. The dial may be a button dial. The user interface may, in response to a press of the button dial, select the item associated with the pre-selection indication to allow editing of a value associated with the parameter represented by the selected item. The editing may include responding to a rotation of the button dial. In response to a touch of the selected item on the touchscreen, the selected item may revert to the item associated with the pre-selection indication and the value associated with the parameter represented by the selected item may remain unchanged.

In some implementations, in response to a touch of an item in the displayed subset other than the selected item on the touchscreen, the user interface may select the touched item to allow editing of a value associated with the parameter represented by the touched item. Editing of the value associated with the parameter represented by the touched item may leave the value associated with the parameter represented by the item associated with the pre-selection indication unchanged. Optionally, in response to the press of the button dial, the user interface may be disabled to swipe gestures on the touchscreen until the value editing may be complete. In some implementations, an operation of the pre-selection indication may permit inputting a control parameter used for controlling operation of the medical therapy device. The control parameter may be applied by the medical therapy device in the operation of a pressure generator to generate a respiratory therapy.

Some versions of the present technology may include a medical therapy device. The medical therapy device may include a user interface. The user interface may include a touchscreen and a dial. The medical therapy device may include a controller coupled with the user interface, and configured to control a therapy operation of the medical therapy device. The controller may be configured to control generation of a display area on the touchscreen. The display area may include a subset of a list of items, each item representing a parameter for the controller to operate the medical therapy device. The display area may include a pre-selection indication in association with an item of the subset. The controller may be configured to detect rotation of the dial, and in response thereto, modify the display area to adjust the association of the pre-selection indication to another item in the display area. The controller may be configured to detect a swipe gesture on the touchscreen and in response thereto, modify the display area to scroll items of the subset while constraining scrolling of the pre-selection indication at a boundary of the display area.

In some implementations, the controller may be further configured to, in response to the detection of the swipe gesture, scroll the pre-selection indication in association with an item of the subset. The controller may be further configured to, in response to the detection of the swipe gesture, adjust the association of the pre-selection indication to another item of the subset at the boundary of the display area.

In some implementations, the dial may be a button dial. The controller may be further configured to detect a press of the button dial, and in response thereto, select the item in association with the pre-selection indication to enable an edit operation on a data value for the parameter represented by the selected item. The controller may be further configured to edit the data value in further response to detection of rotation of the button dial. The controller may be further configured to, in response to detection of a touch of the selected item on the touchscreen, revert the selected item to the item associated with the pre-selection indication and abort a change to the data value associated with the parameter represented by the selected item. The controller may be further configured to, in response to detection of a touch on the touchscreen of an item in the display area other than the pre-selection indication, select the touched item to enable an edit operation on a value associated with the parameter represented by the touched item. The controller may be further configured to, in response to detection of a press of the button dial, disable responding to swipe gestures on the touchscreen until the value editing operation completes.

In some implementations, the controller may be further configured to control an operation of the medical therapy device based on an operation of the pre-selection indication that permits an input of at least one data value for at least one parameter represented by at least one of the items. Optionally, the medical therapy device further may include a pressure generator. The controller may be further configured to apply the at least one data value as a setting for operation of the pressure generator to generate a respiratory therapy.

Some versions of the present technology may include a method of a controller for operating a medical therapy device with a user interface. The method may include controlling generation of a display area on a touchscreen of the medical device. The display area may include a subset of a list of items. The items of list may represent respective parameters for the controller to operate the medical therapy device. The display area may include a pre-selection indication in association with an item of the subset. The method may include detecting rotation of a dial, and in response thereto, modifying the display area to adjust the association of the pre-selection indication to another item in the display area. The method may include detecting a swipe gesture on the touchscreen and in response thereto, modifying the display area to scroll items of the subset while constraining scrolling of the pre-selection indication at a boundary of the display area. The method may include controlling a therapy operation of the medical therapy device.

In some implementations, the method may further include controlling the therapy operation of the medical therapy device based on an operation of the pre-selection indication that permits an input of at least one data value for at least one parameter represented by at least one of the items. The therapy operation may include controlling a pressure generator by applying the at least one data value as a setting for operation of the pressure generator to generate a respiratory therapy.

Some versions of the present technology may include a computer-readable medium having encoded thereon processor control instructions that when executed by a controller of medical therapy device cause the controller to perform any one of more the aspects of a method of operating a user interface as described in more detail herein.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of medical therapy devices.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

FIG. 1 shows a system including a patient 1000 wearing a patient interface 3000, in the form of a full-face mask, receiving a supply of air at positive pressure from an RPT device 4000. Air from the RPT device is conditioned in a humidifier 5000, and passes along an air circuit 4170 to the patient 1000.

FIG. 2 shows an overview of a human respiratory system including the nasal and oral cavities, the larynx, vocal folds, oesophagus, trachea, bronchus, lung, alveolar sacs, heart and diaphragm.

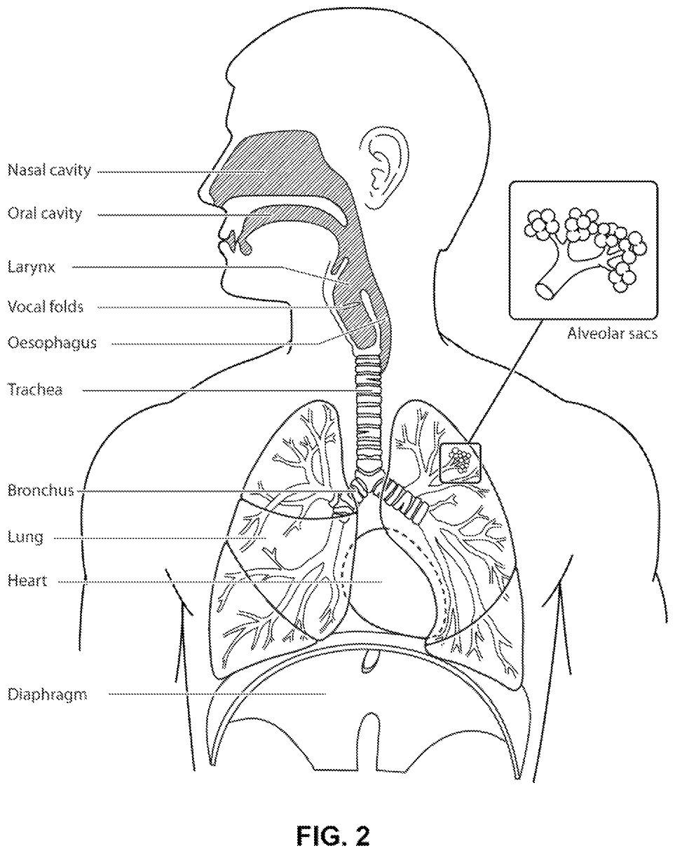

FIG. 3A shows a patient interface in the form of a nasal mask in accordance with one form of the present technology.

FIG. 3B shows a patient 1000 wearing an example of an unsealed patient interface 3800 in the form of a nasal cannula in accordance with one form of the present technology. The patient interface 3800 may be fluidly coupled to receive breathable gas from an RPT device and/or a POC device so as to provide a flow therapy, such as an anti-infection therapy as described in more detail herein.

FIG. 4A shows an RPT device in accordance with one form of the present technology.

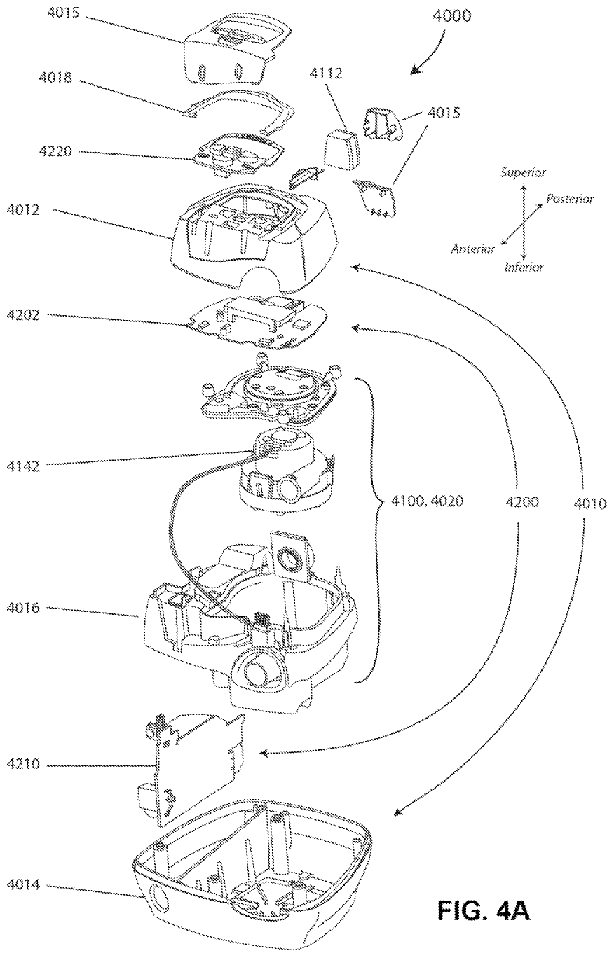

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

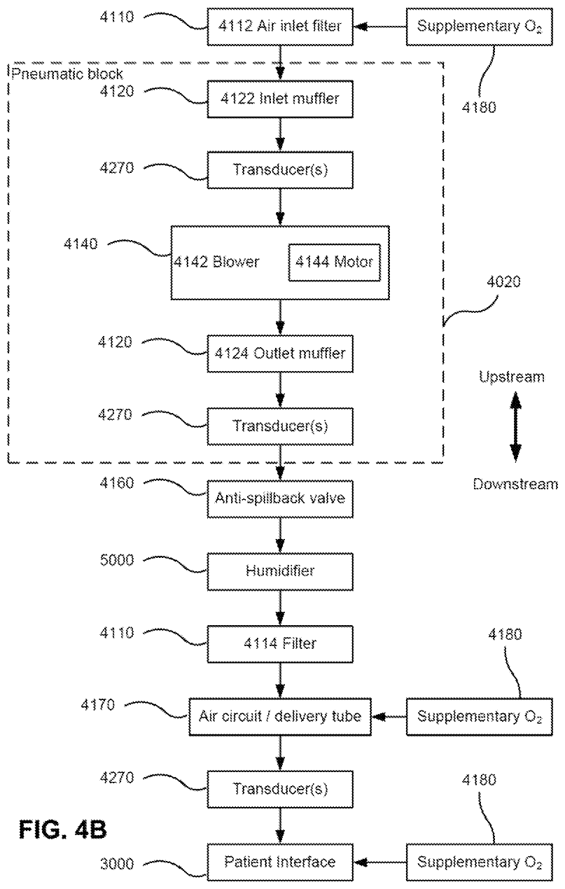

FIG. 4C is a schematic diagram of the electrical components of an RPT device in accordance with one form of the present technology.

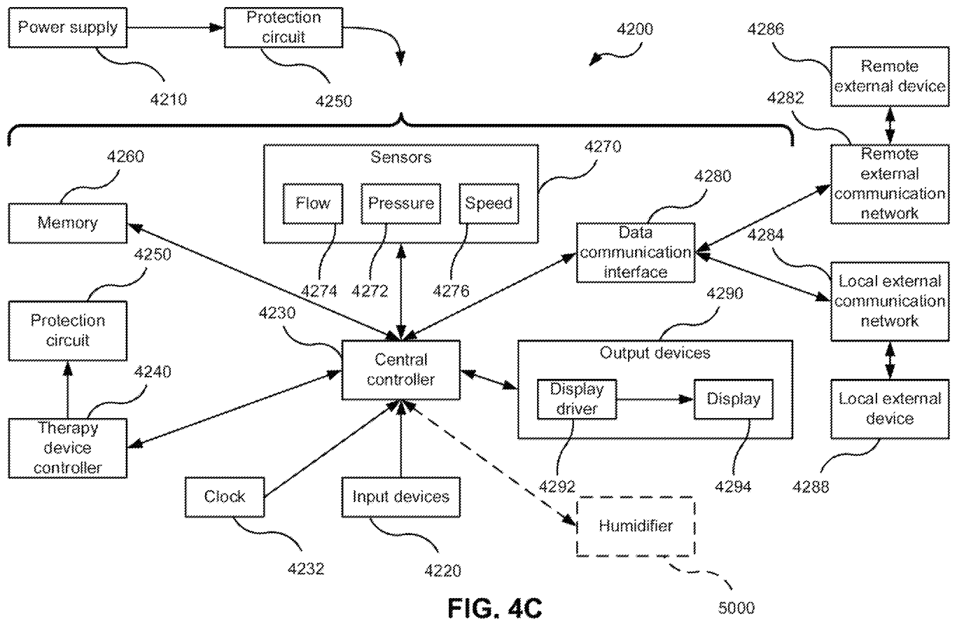

FIG. 5A shows an isometric view of a humidifier in accordance with one form of the present technology.

FIG. 6 is an illustration including a graphic portion of a display and/or user interface for data parameter input to a medical therapy device before any gestures have occurred.

FIG. 7 is an illustration of the display of FIG. 6 after a clockwise turn of a button dial.

FIG. 8 is an illustration of the display of FIG. 7 after a downward swipe gesture.

FIG. 9 is an illustration of the display of FIG. 8 after a large upward swipe gesture.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### Respiratory Therapy Systems

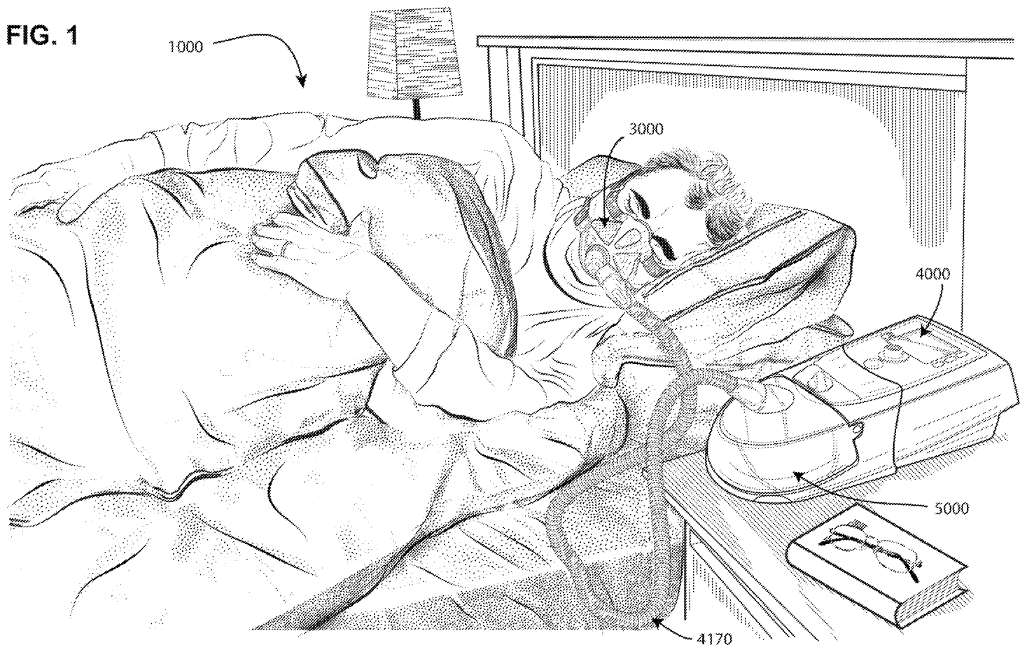

In some example, the present technology may involve a respiratory therapy medical device for treating a respiratory disorder. As illustrated in the example of FIG. 1, a respiratory therapy system may comprise an RT device such as an RPT device 4000, or an HFT device, for supplying a flow of air to the patient 1000 via an air circuit 4170 and a patient interface 3000, 3800. Although described herein in relation to an example RPT device so that the technology may be readily explained, it will be understood that the disclosed user interface technology discussed herein may be applied to other medical devices, including the examples identified herein such as an RT device, an HFT device, a POC device, a humidifier etc.

### 5.1 PATIENT INTERFACE

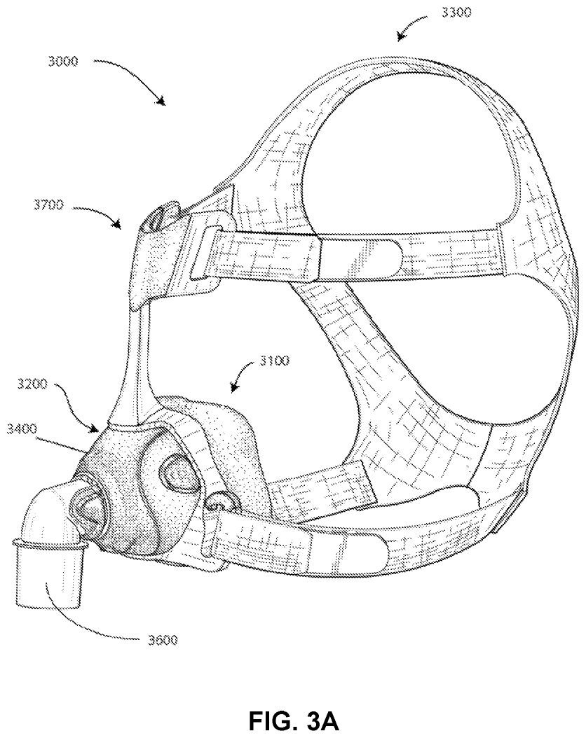

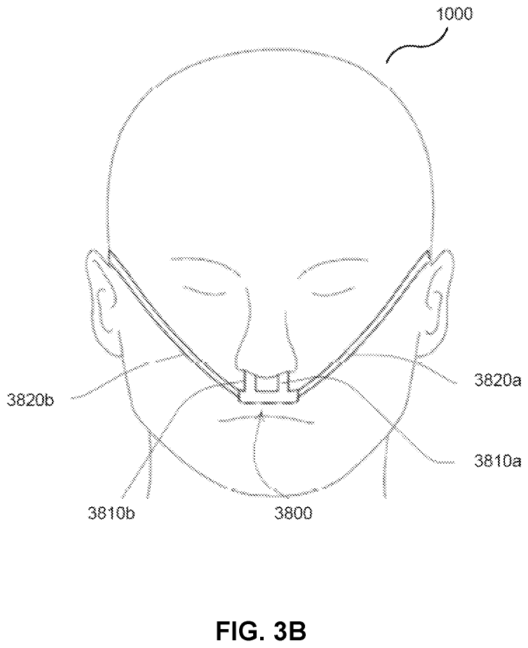

A non-invasive sealed patient interface 3000 as illustrated in the example of FIG. 3A comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use, the seal-forming structure 3100 is arranged to surround an entrance to the airways of the patient so as to facilitate the supply of air at positive pressure to the airways.

An unsealed patient interface 3800, which may be in the form of a nasal cannula, such as one illustrated in FIG. 3B, includes nasal prongs 3810a, 3810b which can deliver air to respective nares of the patient 1000 via respective orifices in their tips. Such nasal prongs do not generally form a seal with the inner or outer skin surface of the nares. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” at the unsealed patient interface 3800, through which excess airflow escapes to ambient, is the passage between the end of the prongs 3810a and 3810b of the nasal cannula-type unsealed patient interface 3800 via the patient's nares to atmosphere.

### 5.2 AIR CIRCUIT

An air circuit 4170 in accordance with one form of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface 3000, 3800. In some forms, there may be separate limbs of the circuit for inhalation and exhalation. In other forms, a single limb circuit is used.

### 5.3 RT DEVICE

An RPT device 4000 comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of delivering a flow of air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.3.1 RPT Device Mechanical & Pneumatic Components

An RPT device may comprise one or more of the following components in an integral unit. In an alternative form, one or more of the following components may be located as respective separate units.

### 5.3.1.1 Air Filter(s)

An RPT device in accordance with one form of the present technology may include an air filter 4110, or a plurality of air filters 4110.

In one form, an inlet air filter 4112, for example an antibacterial filter, is located at the beginning of the pneumatic path upstream of a pressure generator 4140.

In one form, in addition or alternatively to the inlet air filter 4112, an outlet air filter 4114, for example an antibacterial filter, is located between an outlet of the pneumatic block 4020 and a patient interface 3800.

### 5.3.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the Pneumatic Path Between the Pressure Generator 4140 and a Patient Interface 3800.

### 5.3.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers housed in a blower housing, such as in a volute. The blower 4142 may be capable of delivering a supply of air at a controllable flow rate, for example at a rate of up to about 70 to 80 litres per minute. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form, a signal from a transducer 4270 may be filtered, such as by low-pass, high-pass or band-pass filtering.

### 5.3.1.4.1 Flow Rate Sensor

A flow rate sensor 4274 in accordance with the present technology may be based on a differential pressure transducer, for example, an SDP600 Series differential pressure transducer from SENSIRION.

In one form, a signal representing a flow rate of the flow of air at the output of the RPT device 4000 (the device flow rate Qd) is generated by the flow rate sensor 4274.

### 5.3.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal representing a pressure of the flow of air at the output of the RPT device 4000 (the device pressure Pd) is generated by the pressure sensor 4272.

### 5.3.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.3.1.6 Supplementary Gas Delivery

In one form of the present technology, supplementary gas, e.g. oxygen 4180 is delivered to one or more points in the pneumatic path, such as upstream of the pneumatic block 4020, to a point in the air circuit 4170, and/or at the patient interface 3800. Such gas may be provided by a POC, which may optionally include a user interface with features as described in more detail herein.

### 5.3.2 RPT Device Electrical Components

### 5.3.2.1 Power Supply

A power supply 4210 may be located internal or external to the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.3.2.2 Input Devices

In one form of the present technology, an RPT device 4000 includes one or more input devices 4220 in the form of buttons, switches or dials to allow a person to interact with the device. The buttons, switches or dials may be physical devices, or virtual software devices accessible via a touch screen. The buttons, switches or dials may, in one form, be physically connected to the external housing 4010, or may, in another form, be in wireless communication with a receiver that is in electrical connection to the central controller 4230. Additional details for such input devices when serving as part of a user interface may be considered in more detail herein.

In one form, the input device 4220 may be constructed and arranged to allow a person to select a value and/or a menu option.

### 5.3.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000.

The central controller 4230 may be configured to provide output signal(s) to one or more of an output device 4290, a therapy device controller 4240, a data communication interface 4280, and the humidifier 5000.

In some forms of the present technology, the central controller 4230 is configured to execute the one or more methodologies described herein, such as the one or more algorithms which may be implemented with processor-control instructions, expressed as computer programs stored in a non-transitory computer readable storage medium, such as memory 4260. In some forms of the present technology, the central controller 4230 may be integrated with the RPT device 4000.

As such, the central controller or processor(s) described herein may be configured to generate the operations of the user interface(s) described in more detail herein including, for example, generating parameter items on a display and accepting input for adjusting such parameters and/or such a graphic display.

### 5.3.2.4 Clock

The RPT device 4000 may include a clock 4232 that is connected to the central controller 4230.

### 5.3.2.5 Therapy Device Controller

In one form of the present technology, therapy device controller 4240 is a therapy control module 4330 that forms part of the algorithms executed by the central controller 4230.

In one form of the present technology, therapy device controller 4240 is a dedicated motor control integrated circuit. For example, in one form a MC33035 brushless DC motor controller, manufactured by ONSEMI is used.

### 5.3.2.6 Protection Circuits

The one or more protection circuits 4250 in accordance with the present technology may comprise an electrical protection circuit, a temperature and/or pressure safety circuit.

### 5.3.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms described below.

### 5.3.2.8 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet, remote control, or other ancillary device.

### 5.3.2.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.3.2.9.1 Display Driver

A display driver 4292 receives as an input the characters, symbols, or images intended for display on the display 4294, and converts them to commands that cause the display 4294 to display those characters, symbols, or images.

### 5.3.2.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol. Such a display may be a touchscreen and may serve as part of user interface as described in more detail herein.

### 5.3.3 RPT Device Algorithms

As mentioned above, in some forms of the present technology, the central controller 4230 may be configured to execute one or more algorithms expressed as computer programs stored in a non-transitory computer readable storage medium, such as memory 4260. Such algorithms may involve one or more parameter as entered with a user interface as described in more detail herein.

In some forms of the present technology, some portion or all of the algorithms may be implemented by a controller of an external device such as the local external device 4288 or the remote external device 4286. In such forms, data representing the input signals and/or intermediate algorithm outputs necessary for the portion of the algorithms to be executed at the external device may be communicated to the external device via the local external communication network 4284 or the remote external communication network 4282. In such forms, the portion of the algorithms to be executed at the external device may be expressed as computer programs stored in a non-transitory computer readable storage medium accessible to the controller of the external device. Such programs configure the controller of the external device to execute the portion of the algorithms to be executed at the external device.

### 5.4 HUMIDIFIER

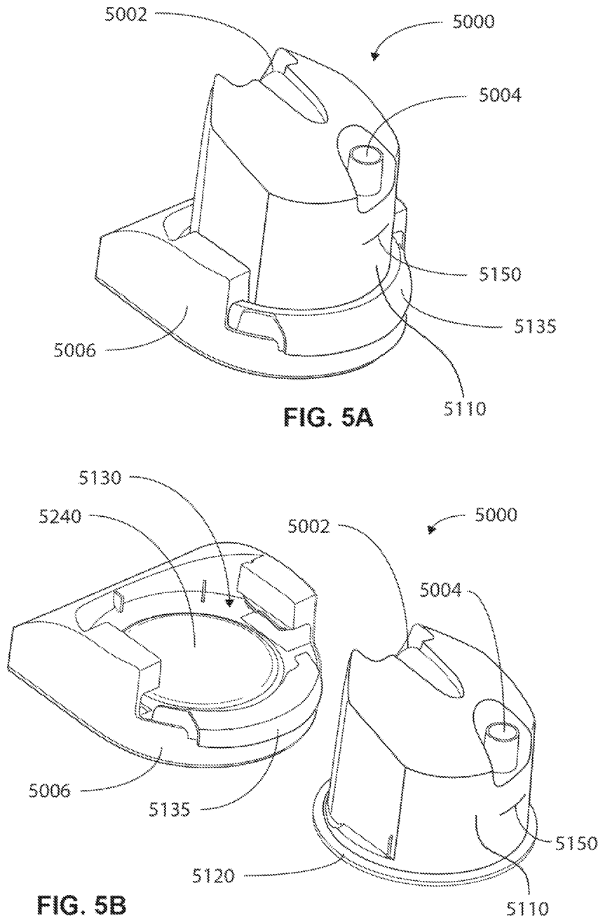

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. Alternatively, a bubble humidifier 5000 may be employed.

### 5.5 MEDICAL THERAPY DEVICE USER INTERFACE

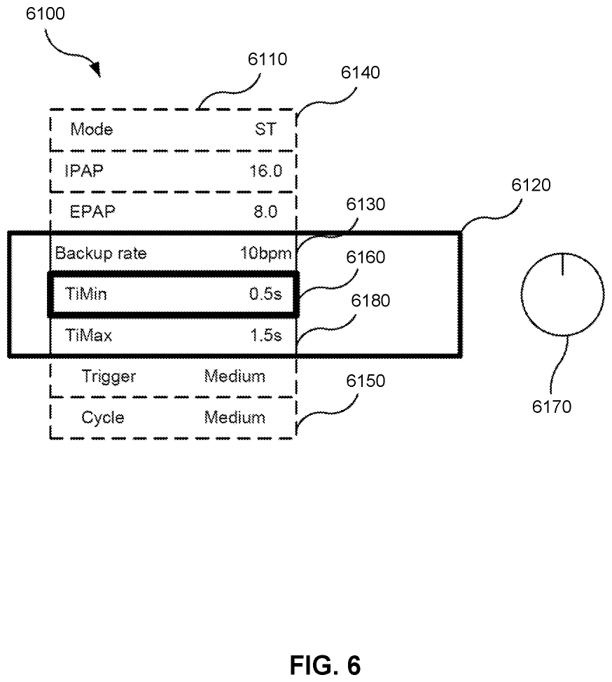

FIG. 6 is an illustration 6100 including an example graphic portion for a display of an example medical therapy device, which in this example is a ventilator. That is, a controller or processor(s) of the medical therapy device (e.g., the RT device) may generate such a graphic display via display interface on a display. The illustration 6100 shows a list 6110 of eight parameter items representing respective parameters for ventilation therapy. Each parameter item in the list, e.g. the parameter item 6140, is displayable in a display area 6120 of a display such as in a frame or rectangle with the name of the parameter (e.g. “Mode” for parameter item 6140) displayed at left and a value for the parameter (e.g. “ST” for parameter item 6140) at the right of the rectangle. In this regard, the display area is a visual presentation on the display. The items of the list 6110 outside the display area (frame 6120) are items of the list that might not be visible on the display since they are out of the display area. The parameter items of the list as well as the values associated with the parameter items may be stored in a memory of the controller or processor in any suitable data structure(s).

The display of the ventilator is a touchscreen whose extent in relation to the list 6110 of parameter items as illustrated in FIG. 6 may be by the thick rectangle representing a display and active touch area of the touchscreen type display 6120. The touchscreen type display 6120 is configured to display a subset 6130 of (e.g., three, four etc.) consecutive parameter items in the list 6110. As illustrated in the example of FIG. 6, the displayed subset 6130 contains three parameter items, respectively named “Backup rate”, “TiMin”, and “TiMax”.

The parameter items preceding the displayed subset 6130 in the list 6110, and therefore not displayed on the touchscreen type display 6120, such as the parameter item 6140 named “Mode”, are illustrated in dashed outline. Likewise, the parameter items following the displayed subset 6130 in the list 6110, and therefore not displayed on the display area of the touchscreen type display 6120, such as the parameter item 6150 named “Cycle”, are illustrated in dashed outline.

In FIG. 6, a dial 6170 is illustrated by a circle that may represent a button dial associated with the touchscreen type display 6120 in a combined touchscreen/dial user interface as described above. Thus, the dial may be a physically rotatable and pressable input device for operation with the processor(s) or controller described herein. The parameter item 6160 named “TiMin”, marked with a doubly-thick outline, is the parameter item currently considered by the controller or processor to be a pre-selected parameter item that is associated with a parameter, which in this example, is an inspiration time minimum threshold parameter. Thus, the controller may generate a pre-selection indication on the display as an indication of such a pre-selected parameter item (e.g., the doubly thick outline or other visible demarcation.) As such, the processor(s) or controller may access one or more data structure(s) in a memory to maintain an accessible association between the pre-selection indication and the pre-selected item of the list, and/or its parameter, to achieve the operational functionality of the user interface and its display as described herein. For example, if the controller or processor detects a press of the button dial 6170, it will operate the display to select the parameter item 6160 that is the pre-selected item and therefore its associated parameter, and allow the user to edit the current value for that parameter. In some implementations of the present technology, once the pre-selected parameter item 6160 is selected for value editing, turning the button dial 6170 does not alter the pre-selected parameter item on the display, but rather plays a role in the editing of the parameter value with the display.

In some implementations of the present technology, the user can exit such a “value editing” mode without altering the value of the parameter being edited, e.g. the parameter associated with parameter item 6160, by simply touching the item representing the parameter item 6160 on the touchscreen. This touch operation, when detected by the controller or processor, returns the selected parameter item 6160 to pre-selection status as illustrated in FIG. 6.

In some implementations of the present technology, while in “value editing” mode, the user can switch to editing another parameter item in the displayed subset 6130, e.g. the parameter item 6180 named “Timax”, without altering the value of the associated parameter previously being edited, e.g. the parameter item 6160, by simply touching another parameter item 6180 representing the other parameter on the touchscreen.

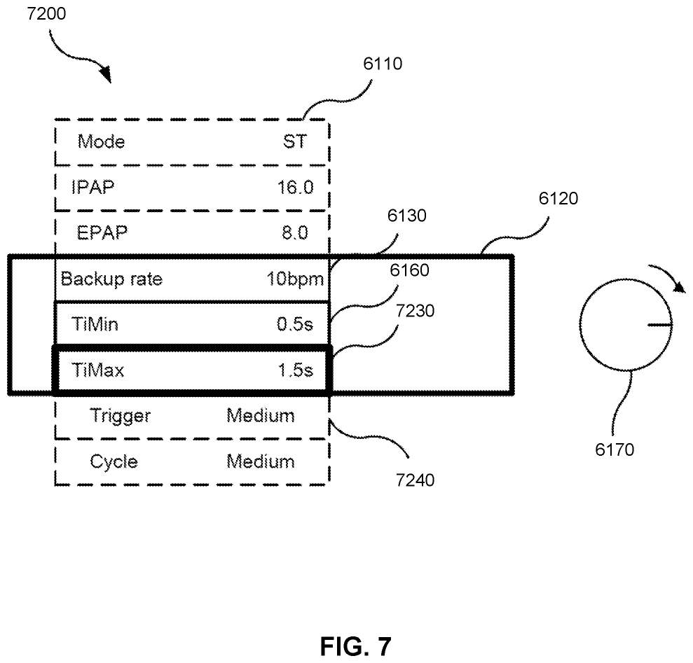

FIG. 7 is an illustration 7200 of a graphic portion of the display of the device generated by the controller or processor after the controller or processor detects a clockwise turn of the button dial 6170 such as by a single predetermined angular increment. The parameter item 7230 named “TiMax”, immediately below the parameter item 6160 named “Timin” in the list 6110, is now pre-selected as a result of the turn, but the displayed subset 6130 of the list 6110 is the same as in the illustration 6100.

If the controller or processor(s) detects that the button dial 6170 is turned through a further clockwise angular increment, the display is generated so that the parameter item 7240 named “Trigger” would become the pre-selected item. To allow the pre-selected item to remain part of the displayed subset, the display is generated so that the displayed subset 6130 would scroll down by one item so that the new displayed subset consisted of the parameter items named “TiMin”, “TiMax”, and “Trigger” and the parameter item named “Backup rate” would be “scrolled out” such as to be removed from the display area.

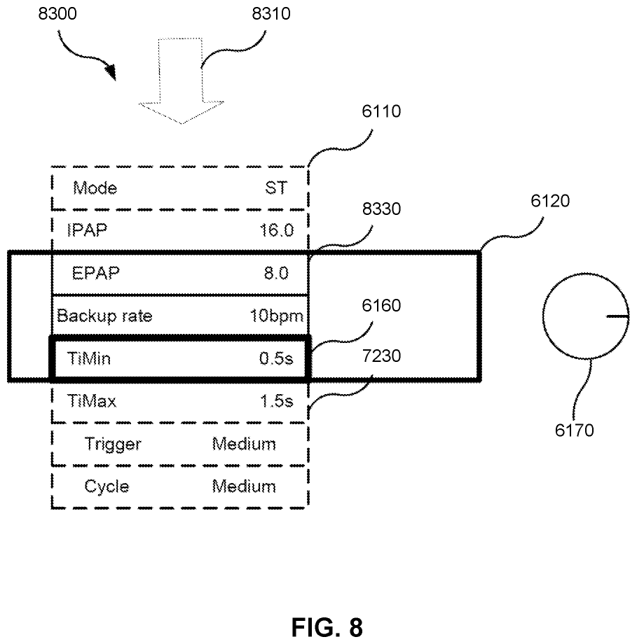

FIG. 8 is an illustration 8300 of a graphic portion of the display that is generated on the device after the controller or processor detects that a downward “swipe” gesture 8310, as denoted by the arrow at 8310, on an active touch area of the touchscreen type display 6120 from the graphic state illustrated as 7200 in FIG. 7. The displayed subset 8330 is generated so that it has effectively moved (scrolled) upwards by one item of the list 6110 compared to the displayed subset 6130 in response to the downward swipe gesture 8310 and now comprises the parameter items named “EPAP”, “Backup Rate”, and “TiMin”. The previously pre-selected parameter item 7230, named “TiMax”, is no longer generated as part of the displayed subset 8330 and has moved beyond the lower boundary of the display area touchscreen 6120. If such an undisplayed parameter item 7230 remained the pre-selected parameter item, this could potentially cause the user some confusion as they would no longer be able to tell which parameter would be selected if they were to press the button dial 6170. However, in accordance with the present technology, the controller or processor(s) control the display so that the pre-selection effectively changes (e.g., moves upwards) in the list 6110 to the lowermost parameter in the new displayed subset 8330, so that the parameter item 6160 named “TiMin” is now the pre-selected parameter item. In other words, the controller may operate the pre-selection indicator in relation to the display and the items of the list so that the pre-selection indication has an appearance of being “pushed against” or constrained by the lower boundary of display area of the touchscreen type display 6120 rather than being allowed to disappear beyond the lower boundary of the touchscreen 6120 as a result of the downward swipe/upward scroll. Thus, the controller may generate the display in response to a swipe gesture (either up or down) such that the pre-selected item with the pre-selection indication may scroll with the items of the list with no change to the pre-selected item until the pre-selected item arrives at a boundary of the display area, at which time the pre-selected item will change to another displayed item(s), as indicated with the pre-selection indication, such as when the items of the list continue to scroll through the display area as a result of the swipe gesture. In this regard, the boundary of the display area (e.g., upper or lower) of the display may serve to change the pre-selected item in the display as shown by the displayed pre-selection indication, so as to maintain the pre-selection indication on some item that is still within a given display area of the display.

In one implementation of the present technology, when a press of the button dial 6170 to select the pre-selected parameter item 6160 for value editing is detected by the controller or processor(s), the controller operates the touchscreen so that swipe gestures are disabled or disregarded by the controller, until the editing is complete. This helps to ensure that the parameter that is being value-edited cannot be “scrolled out” of the display area (the displayed subset 6130) and thereby out of sight of the user during the editing operation.

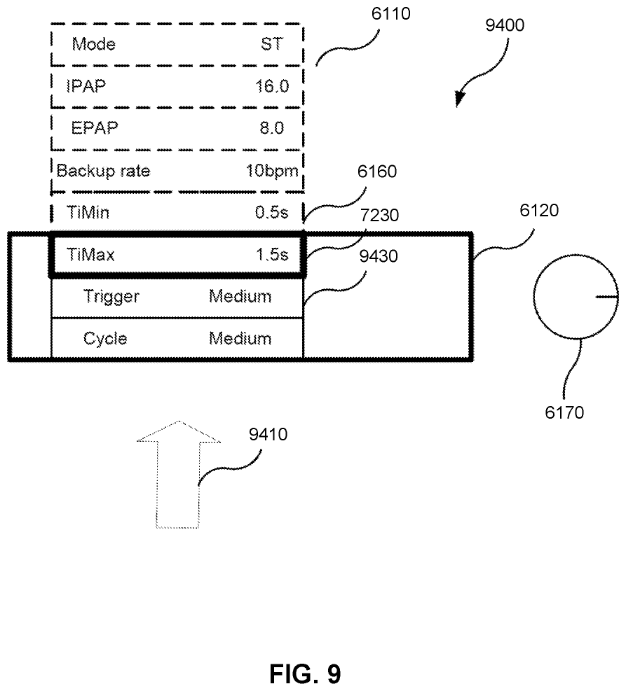

FIG. 9 is an illustration 9400 of the display of the device after an upward “swipe” gesture, as denoted by the arrow 9410, on the touchscreen 6120 from the state illustrated as 8300 in FIG. 8. Based on the detection of the extent of the swipe, the displayed subset 9430 may be generated on the display by the controller or processor(s) so that the items of the list pass through the display area such that it has effectively moved (scrolled) downwards by three items through the list 6110 in response to the upward swipe. The display area now comprises the parameter items named “TiMax”, “Trigger”, and “Cycle”. Moreover, the previously pre-selected parameter item 6160, named “TiMin”, is no longer part of the displayed subset 9430 and has scrolled out of the display area so as to be seemingly beyond the upper boundary of the touchscreen 6120. If the now undisplayed parameter 6160 remained the pre-selected parameter, this could potentially cause the user some confusion as they would no longer be able to predict which parameter would be selected if they were to press the button dial 6170. However, in accordance with the present technology as previously described, the pre-selection indication is moved within and maintained in the display area such that it is displayed as moving to the upward boundary of the display area such as from the downward boundary but then changing its pre-selection item by moving downwards in the list 6110 so as to pre-select the uppermost parameter item in the displayed subset 9430 when constrained by the display area boundary. As illustrated in the example, the parameter item 7230 named “TiMax” is then generated on the display as the pre-selected parameter with the pre-selection indication. In other words, the pre-selection has “pushed against” the upper boundary of the touchscreen 6120 rather than being allowed to disappear beyond the upper boundary of the touchscreen 6120 as a result of the upward swipe/downward scroll.

It may be seen therefore from FIGS. 8 and 9 that according to the combined dial/touchscreen user interface of the present technology, the pre-selection “pushes against” the boundary of the touchscreen in the direction of a swipe gesture such that the pre-selected item remains part of the displayed subset. Absent such pushing, the resultant scroll may cause the pre-selection to move outside the displayed subset, potentially confusing the user.

### 5.6 GLOSSARY

For purposes of the present technology disclosure, in certain forms of the present technology, one or more of the following definitions may apply. In other forms of the present technology, alternative definitions may apply.

### 5.6.1 General

Air: In certain forms of the present technology, air supplied to a patient may be atmospheric air, and in other forms of the present technology atmospheric air may be supplemented with oxygen.

Continuous Positive Airway Pressure (CPAP): CPAP treatment will be taken to mean the application of a supply of air or breathable gas to the entrance to the airways at a pressure that is continuously positive with respect to atmosphere, and preferably approximately constant through a respiratory cycle of a patient. In some forms, the pressure at the entrance to the airways will vary by a few centimetres of water within a single respiratory cycle, for example being higher during inhalation and lower during exhalation. In some forms, the pressure at the entrance to the airways will be slightly higher during exhalation, and slightly lower during inhalation. In some forms, the pressure will vary between different respiratory cycles of the patient, for example being increased in response to detection of indications of partial upper airway obstruction, and decreased in the absence of indications of partial upper airway obstruction.

### 5.6.2 Aspects of PAP Devices

Air circuit: A conduit or tube constructed and arranged in use to deliver a supply of air or breathable gas between a PAP device and a patient interface. In particular, the air circuit may be in fluid connection with the outlet of the pneumatic block and the patient interface. The air circuit may be referred to as air delivery tube. In some cases there may be separate limbs of the circuit for inhalation and exhalation. In other cases a single limb is used.

APAP: Automatic Positive Airway Pressure. Positive airway pressure that is continually adjustable between minimum and maximum limits, depending on the presence or absence of indications of SDB events.

Blower or flow generator: A device that delivers a flow of air at a pressure above ambient pressure.

Controller: A device, or portion of a device that adjusts an output based on an input. For example, one form of controller has a variable that is under control—the control variable—that constitutes the input to the device. The output of the device is a function of the current value of the control variable, and a set point for the variable. A servo-ventilator may include a controller that has ventilation as an input, a target ventilation as the set point, and level of pressure support as an output. Other forms of input may be one or more of oxygen saturation (SaO₂), partial pressure of carbon dioxide (PCO₂), movement, a signal from a photoplethysmogram, and peak flow. The set point of the controller may be one or more of fixed, variable or learned. For example, the set point in a ventilator may be a long term average of the measured ventilation of a patient. Another ventilator may have a ventilation set point that changes with time. A pressure controller may be configured to control a blower or pump to deliver air at a particular pressure.

Therapy: Therapy in the present context may be one or more of positive pressure therapy, oxygen therapy, carbon dioxide therapy, control of dead space, and the administration of a drug.

Motor: A device for converting electrical energy into rotary movement of a member. In the present context the rotating member is an impeller, which rotates in place around a fixed axis so as to impart a pressure increase to air moving along the axis of rotation.

Positive Airway Pressure (PAP) device: A device for providing a supply of air at positive pressure to the airways.

Transducers: A device for converting one form of energy or signal into another. A transducer may be a sensor or detector for converting mechanical energy (such as movement) into an electrical signal. Examples of transducers include pressure sensors, flow sensors, carbon dioxide (CO₂) sensors, oxygen (O₂) sensors, effort sensors, movement sensors, noise sensors, a plethysmograph, and cameras.

Volute: The casing of the centrifugal pump that receives the air being pumped by the impeller, slowing down the flow rate of air and increasing the pressure. The cross-section of the volute increases in area towards the discharge port.

### 5.6.3 Aspects of the Respiratory Cycle

Apnea: An apnea will be said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Preferably, flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) Chair-shaped: Having a single local peak, the peak being at the leading edge, followed by a relatively flat portion.
(iii) Reverse-chair shaped: Having a relatively flat portion followed by single local peak, the peak being at the trailing edge.
(iv) M-shaped: Having two local peaks, one at the leading edge, and one at the trailing edge, and a relatively flat portion or a dip between the two peaks.

Hypopnea: A hypopnea will be taken to be a reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal flow.

Inspiratory portion of a breathing cycle: Preferably the period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow (Qpeak): The maximum value of flow during the inspiratory portion of the respiratory flow waveform.

Respiratory flow, airflow, patient airflow, respiratory airflow (Qr): These synonymous terms may be understood to refer to the PAP device's estimate of respiratory airflow, as opposed to “true respiratory flow” or “true respiratory airflow”, which is the actual respiratory flow experienced by the patient, usually expressed in litres per minute.

Tidal volume (Vt): The volume of air inhaled or exhaled during normal breathing, when extra effort is not applied.

(inhalation) Time (Ti): The duration of the inspiratory portion of the respiratory flow waveform.

(exhalation) Time (Te): The duration of the expiratory portion of the respiratory flow waveform.

(total) Time (Ttot): The total duration between the start of the inspiratory portion of one respiratory flow waveform and the start of the inspiratory portion of the following respiratory flow waveform.

Upper airway obstruction (UAO): includes both partial and total upper airway obstruction. This may be associated with a state of flow limitation, in which the level of flow increases only slightly or may even decrease as the pressure difference across the upper airway increases (Starling resistor behaviour).

Ventilation (Vent): A measure of the total amount of gas being exchanged by the patient's respiratory system, including both inspiratory and expiratory flow. When expressed as a volume per minute, this quantity is often referred to as “minute ventilation”. Minute ventilation is sometimes given simply as a volume, understood to be the volume per minute.

### 5.6.4 PAP Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow will be given the symbol Q. Total flow, Qt, is the flow of air leaving the PAP device. Vent flow, Qv, is the flow of air leaving a vent to allow washout of exhaled gases. Leak flow, Ql, is the flow rate of unintentional leak from a patient interface system. Respiratory flow, Qr, is the flow of air that is received into the patient's respiratory system.

Leak: A flow of air to the ambient. Leak may be intentional, for example to allow for the washout of exhaled CO₂. Leak may be unintentional, for example, as the result of an incomplete seal between a mask and a patient's face.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. For nasal CPAP treatment of OSA, a reference to treatment pressure is a reference to a pressure in the range of about 4-20 cmH₂O, or about 4-30 cmH₂O. The pressure in the patient interface (or, more succinctly, mask pressure) is given the symbol Pm.

### 5.6.5 Terms for Ventilators

Adaptive Servo-Ventilator: A ventilator that has a changeable, rather than fixed target ventilation. The changeable target ventilation may be learned from some characteristic of the patient, for example, a respiratory characteristic of the patient.

Backup rate: a parameter of a ventilator that establishes the minimum respiration rate (typically in number of breaths per minute) that the ventilator will deliver to the patient, if not otherwise triggered.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

EPAP (or EEP): a base pressure, to which a pressure varying within the breath is added to produce the desired mask pressure which the ventilator will attempt to achieve at a given time.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the minimum value during expiration (e.g., PS=IPAP−EPAP). In some contexts pressure support means the difference which the device aims to achieve, rather than what it actually achieves.

Servo-ventilator: A ventilator that measures patient ventilation has a target ventilation, and which adjusts the level of pressure support to bring the patient ventilation towards the target ventilation.

Spontaneous/Timed (S/T)—A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

Ventilator: A mechanical device that provides pressure support to a patient to perform some or all of the work of breathing.

Ventilator inspiration and ventilator expiration: the periods during which the ventilator considers that it should deliver pressures appropriate respectively to patient inspiration and expiration. Depending on the quality of patient-ventilator synchronisation, and the presence of upper airway obstruction, these may or may not correspond to actual patient inspiration or expiration.

### 5.7 OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

The various methods or processes outlined herein for generation of the display area of user interface in response to user interaction therewith may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various aspects of the aforementioned technology functional concepts may be embodied as processor control instructions on a processor readable medium or computer readable storage medium (or multiple such storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs or processor control instructions that, when executed on one or more computers, controller or other processors, perform methods that implement the various embodiments of the technology discussed herein. The computer readable medium or media can be transportable, such that the program or programs stored thereon can be loaded onto one or more different computers or other processors to implement various aspects of the present technology as discussed above.

The terms “program” or “software” are used herein in a generic sense to refer to any type of computer code or set of computer-executable instructions that can be employed to program a computer, controller, or other processor to implement various aspects of embodiments as discussed above. Additionally, it should be appreciated that according to one aspect, one or more computer programs that when executed perform methods of the present technology need not reside on a single computer, controller or processor, but may be distributed in a modular fashion amongst a number of different computers, controllers or processors to implement various aspects of the present technology. For example, some versions of the present technology may involve a server with access to any of the computer readable or processor-readable mediums as described herein. The server may be configured to receive requests for downloading the processor-control instructions or processor-executable instructions of the medium to an electronic device, such as a medical device or smart mobile processing device for use with the medical device, such as over a network such as a communications network, an internet or the Internet. Thus, the electronic device may also include such a medium to execute the instructions of the medium. Similarly, the present technology may be implemented as a method of a server having access to any of the mediums described herein. The method(s) may include receiving, at the server, a request for downloading the processor-executable instructions of the medium to a medical device over the network; and transmitting the instructions of the medium to the medical device in response to the request. Optionally, the server may have access to the medium to execute the instructions of the medium.

Computer-executable instructions may be in many forms, such as program modules, executed by one or more computers or other devices. Generally, program modules include routines, programs, objects, components, data structures, etc. that perform particular tasks or implement particular data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. For example, although the scrolling and swiping herein is generally described in relation to up or down directions of the touchscreen, it will be understood that such scrolling or swiping may alternatively be implemented so as to permit scrolling and swiping in left or right directions of a touchscreen to yield a corresponding display control of the parameter items and pre-selection indication, including the boundary related pushing, in the display area in association with these alternative directions. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.8 REFERENCE SIGNS LIST

patient 1000
patient interface 3000
seal-forming structure 3100
plenum chamber 3200
structure 3300
vent 3400
connection port 3600
forehead support 3700
unsealed patient interface 3800
nasal prong 3810a
nasal prong 3810b
lumen 3820a
lumen 3820b
RPT device 4000
external housing 4010
upper portion 4012
portion 4014
panel 4015
chassis 4016
handle 4018
pneumatic block 4020
air filter 4110
inlet air filter 4112
outlet air filter 4114
muffler 4120
inlet muffler 4122
outlet muffler 4124
pressure generator 4140
blower 4142
motor 4144
anti-spill back valve 4160
air circuit 4170
oxygen 4180
electrical components 4200
PCBA 4202
power supply 4210
input device 4220
central controller 4230
clock 4232
therapy device controller 4240
protection circuits 4250
memory 4260
transducers 4270
pressure sensor 4272
flow rate sensor 4274
motor speed transducer 4276
data communication interface 4280
remote external communication network 4282
local external communication network 4284
remote external device 4286
local external device 4288
output device 4290
display driver 4292
display 4294
therapy control module 4330
humidifier 5000
humidifier inlet 5002
humidifier outlet 5004
humidifier base 5006
humidifier reservoir 5110
heating element 5240
illustration 6100
list 6110
touchscreen 6120
subset 6130
parameter item 6140
parameter item 6150
parameter item 6160
button dial 6170
parameter item 6180
illustration 7200
parameter item 7230
parameter item 7240
illustration 8300
downward swipe gesture 8310
displayed subset 8330
illustration 9400
upward swipe gesture 9410
displayed subset 9430

1. A user interface for a medical therapy device, the user interface comprising:
a touchscreen configured to display a subset of a list of items, each item representing a parameter to operate the medical therapy device, wherein one item in the displayed subset is associated with a pre-selection indication, and
a dial configured to be rotated, wherein the pre-selection indication moves from an item to an adjacent item on the displayed subset in response to a rotation of the dial by a predetermined angular increment,
wherein in response to a swipe gesture on the touchscreen:
the displayed subset scrolls through the list, and
the pre-selection indication pushes against a boundary of the touchscreen in the direction of the swipe gesture such that the item associated with the pre-selection indication remains part of the displayed subset.

2. The user interface of claim 1, wherein the pre-selection indication pushes against an upper boundary of the touchscreen in response to an upward swipe gesture.

3. The user interface of any one of claims 1 to 2, wherein the pre-selection indication pushes against the lower boundary of the touchscreen in response to a downward swipe gesture.

4. The user interface of any one of claims 1 to 3, wherein the dial is a button dial.

5. The user interface of claim 4, further comprising, in response to a press of the button dial, selecting the item associated with the pre-selection indication to allow editing of a value associated with the parameter represented by the selected item.

6. The user interface of claim 5, wherein the editing comprises responding to a rotation of the button dial.

7. The user interface of any one of claims 5 to 6, wherein in response to a touch of the selected item on the touchscreen, the selected item reverts to the item associated with the pre-selection indication and the value associated with the parameter represented by the selected item remains unchanged.

8. The user interface of any one of claims 5 to 7, further comprising, in response to a touch of an item in the displayed subset other than the selected item on the touchscreen, selecting the touched item to allow editing of a value associated with the parameter represented by the touched item.

9. The user interface of claim 8, wherein editing of the value associated with the parameter represented by the touched item leaves the value associated with the parameter represented by the item associated with the pre-selection indication unchanged.

10. The user interface of any one of claims 5 to 9, further comprising, in response to the press of the button dial, disabling swipe gestures on the touchscreen until the value editing is complete.

11. The user interface of any one of claims 1 to 10 wherein an operation of the pre-selection indication permits inputting a control parameter used for controlling operation of the medical therapy device.

12. The user interface of claim 11 wherein the control parameter is applied by the medical therapy device in the operation of a pressure generator to generate a respiratory therapy.

13. A medical therapy device comprising:
a user interface, the user interface comprising a touchscreen and a dial; and
a controller coupled with the user interface, and configured to control a therapy operation of the medical therapy device;
the controller configured to:
control generation of a display area on the touchscreen, the display area comprising a subset of a list of items, the items representing respective parameters for the controller to operate the medical therapy device, the display area comprising a pre-selection indication in association with an item of the subset;
detect rotation of the dial, and in response thereto, modify the display area to adjust the association of the pre-selection indication to another item in the display area; and
detect a swipe gesture on the touchscreen and in response thereto, modify the display area to scroll items of the subset while constraining scrolling of the pre-selection indication at a boundary of the display area.

14. The medical therapy device of claim 13, wherein the controller is further configured to, in response to the detection of the swipe gesture, scroll the pre-selection indication in association with an item of the subset.

15. The medical therapy device of any one of claims 13 to 14, wherein the controller is further configured to, in response to the detection of the swipe gesture, adjust the association of the pre-selection indication to another item of the subset at the boundary of the display area.

16. The medical therapy device of any one of claims 13 to 15, wherein the dial is a button dial.

17. The medical therapy device of claim 16, wherein the controller is further configured to detect a press of the button dial, and in response thereto, select the item in association with the pre-selection indication to enable an edit operation on a data value for the parameter represented by the selected item.

18. The medical therapy device of claim 17, wherein the controller is further configured to edit the data value in further response to detection of rotation of the button dial.

19. The medical therapy device of any one of claims 17 to 18, wherein the controller is further configured to, in response to detection of a touch of the selected item on the touchscreen, revert the selected item to the item associated with the pre-selection indication and abort a change to the data value associated with the parameter represented by the selected item.

20. The medical therapy device of any one of claims 17 to 19, wherein the controller is further configured to, in response to detection of a touch on the touchscreen of an item in the display area other than the pre-selection indication, select the touched item to enable an edit operation on a value associated with the parameter represented by the touched item.

21. The medical therapy device of any one of claims 17 to 20, wherein the controller is further configured to, in response to detection of a press of the button dial, disable responding to swipe gestures on the touchscreen until the value editing operation completes.

22. The medical therapy device of any one of claims 13 to 21, wherein the controller is further configured to control an operation of the medical therapy device based on an operation of the pre-selection indication that permits an input of at least one data value for at least one parameter represented by at least one of the items.

23. The medical therapy device of claim 22, further comprising a pressure generator, wherein the controller is further configured to apply the at least one data value as a setting for operation of the pressure generator to generate a respiratory therapy.

24. A method of a controller for operating a medical therapy device with a user interface comprising:
controlling generation of a display area on a touchscreen of the medical device, the display area comprising a subset of a list of items, each item representing a parameter for the controller to operate the medical therapy device, the display area comprising a pre-selection indication in association with an item of the subset;
detecting rotation of a dial, and in response thereto, modifying the display area to adjust the association of the pre-selection indication to another item in the display area,
detecting a swipe gesture on the touchscreen and in response thereto, modifying the display area to scroll items of the subset while constraining scrolling of the pre-selection indication at a boundary of the display area; and
controlling a therapy operation of the medical therapy device.

25. The method of claim 24 further comprising controlling the therapy operation of the medical therapy device based on an operation of the pre-selection indication that permits an input of at least one data value for at least one parameter represented by at least one of the items.

26. The method of claim 25, wherein the therapy operation comprises controlling a pressure generator by applying the at least one data value as a setting for operation of the pressure generator to generate a respiratory therapy.

27. A computer-readable medium having encoded thereon processor control instructions that when executed by a controller of medical therapy device cause the controller to perform the method of any one of claims 24 to 26.
